(19) United States
(12) Patent Application Publication
(10) Pub. No.: US 2025/0176926 A1
(43) Pub. Date: Jun. 5, 2025

(54) Self-Navigating Overhead Support System and Method for Imaging System

(71) Applicant: GE Precision Healthcare LLC, Waukesha, WI (US)

(72) Inventors: Minghui Ye, Beijing (CN); Yan'Nan Huang, Beijing (CN)

(21) Appl. No.: 18/529,366

(22) Filed: Dec. 5, 2023

(51) Int. Cl. A61B 6/00

(52) U.S. Cl. CPC A61B 6/4464; A61B 6/4452; A61B 6/4476

(57) ABSTRACT

An imaging system includes an overhead support system mounted within an environment for the imaging system, an imaging device mounted to the overhead support system, visual and non-visual sensors disposed on the imaging device, a motion controller operably connected to the overhead support system, a processor operably connected to the motion controller and the visual and non-visual sensors to send and receive data signals from the motion controller, and the visual and non-visual sensors, and a memory operably connected to the processor and storing instructions for a self-navigating and positioning system that generates a three-dimensional (3D) map of the environment with data from the visual and non-visual sensors and position data from the motion controller to navigate the overhead support system within the environment from a start position to a finish position to avoid collisions with one or more objects within the environment.

### FIELD OF THE DISCLOSURE

The present disclosure relates to X-ray systems, and more particularly to X-ray systems including overhead support systems employed to move a part of the imaging system, such as the X-ray tube, to accommodate various patient positions.

### BACKGROUND OF THE DISCLOSURE

A number of X-ray imaging systems of various designs are known and are presently in use. Such systems are generally based upon generation of X-rays that are directed toward a subject of interest. The X-rays traverse the subject and impinge on a detector, for example, a film, an imaging receptor, or a portable cassette. The detector detects the X-rays, which are attenuated, scattered or absorbed by the intervening structures of the subject. In medical imaging contexts, for example, such systems may be used to visualize the internal structures, tissues and organs of a subject for the purpose screening or diagnosing ailments.

X-ray systems may be fixed or mobile. Fixed radiography systems generally utilize an X-ray source moveably mounted to ceiling in the area in which the X-rays are to be obtained. In one prior art configuration, the radiography system is an overhead support X-ray system 100 is shown in FIG. 1. The overhead support system 100, or in an exemplary embodiment, the overhead tube support (OTS) system 100 typically includes a column 105 to which the X-ray source 110 is attached, coupled to an overhead rectangular bridge 115 that travels along a system of rails or tubes 120 oriented perpendicular to the bridge 115. A transport mechanism 125 coupled to the bridge 115 operates to move the column 105 along a longitudinal horizontal axis, while the rail system 120 allows the bridge 115 to travel along a lateral horizontal axis in the same plane. The rail system 120 typically includes a front rail 120a, a rear rail 120b, and a cable drape rail (not shown) mounted to a ceiling of a room or suite housing the fixed radiography system. In some installations, the overhead tube support system 100 may be mounted to a system of struts which are fixed to the ceiling to enable the X-ray source 110 to be oriented with respect to a fixed table 130 or fixed wall stand 135 that hold a detector 140 thereon in order to obtain desired images of the patient position on or adjacent thereto.

One environment in which the OTS system 100 can be employed is in a trauma site(s) 200, an exemplary embodiment of which is represented in FIG. 1, such as an emergency room area within a hospital. The trauma site 200 typically includes a number of treatment rooms 202 separated by shortened or half walls 204 and each including one or more stretchers or patient tables 130 therein, along with other monitoring device(s) and/or medical equipment 208 to facilitate treatment of patients within the rooms 202. To accommodate imaging procedures to be performed in each of the rooms 202, the OTS system 100 is constructed with the rails 120 extending over the shortened walls 204 to allow the movement of the carriage or bridge 115 into the selected room 202 in which the imaging procedure is to be performed. Once positioned within the selected room 202, the OTS system 100 can be operated to locate the X-ray source 110 where desired for obtaining the images of the patient on the table 130 i.e., allowing the OTS system 100 to move the X-ray source 110 above the or table 130 on which the patient is positioned.

To effectively treat patients in each of the rooms 202, it is necessary to efficiently be able to move the X-ray source 110 to the desired imaging locations within and between each room 202. As a result, the motion of the OTS system 100 is the greatest concern in terms of efficiency since the OTS system 100 is “shared” by several rooms 202 within the trauma site 200. Without moving smoothly and effectively to reach the desired target position(s) in the room(s) 202, the efficiency of the imaging processes performed by the OTS system 100 and X-ray source 110 can be less than desired. However, while attempts are made to standardize the rooms 202 within the trauma sites 200 to provide the same level of care in each room 202, as a result of the urgent nature of the medical issues treated in the rooms 202, the rooms 202 often contain varying types and amounts of medical equipment 208 therein, with different positioning of the medical equipment 208 within the respective rooms 202 to best preform the necessary treatment of the patient. With this situation, the movement of the OTS system 100 and the X-ray source 110 must be tailored for each room 202 to accommodate the different positions of table 130, medical equipment 208 and medical personnel within the individual rooms 202 to minimize the risk of collision with the OTS system 100 and X-ray source 110 when moving within a particular room 202.

To attempt to address the issues of minimizing the potential for collision of the OTS system 100 and X-ray source 110 with one or more items in the trauma rooms 202 while maximizing the efficiency of the movement of the OTS system 100 and x-ray source 110 within the room 202, two different solutions are currently employed. First, for each room 202 the OTS system 100 is programmed with a predefined route or path between a defined start or park/non-use position and a pre-determined position of the stretcher/table 130 in each room 202. Because the start or park position and the desired position of the x-ray source 110 adjacent the table 130 are predetermined for a selected imaging procedure, the items located within the room 202, i.e., the table 130, the medical equipment 208 and/or medical personnel, can be positioned outside of the path of travel for the OTS system 100 and X-ray source 110 to avoid collisions.

However, a significant drawback to this solution is the requirement that all items in the room 202 be positioned outside of the predetermined path of the OTS system 100 and X-ray source 110, which can often and easily be overlooked as a results of the urgent nature of the medical issues being treated within the room 202, and the resulting changeable positioning of the items within the rooms 202. In particular, while the intended path of the OTS system 100 and the X-ray source 110 is known by the OTS system 100, it may not be known by medical personnel in the room 202. Further, while the OTS system 100 can also mitigate the situations where a collision does occur by limiting the collision force and/or shutting down the drive mechanism for the OTS system 100, this solution to the avoidance of the collision between the OTS system 100 and items within the rooms 202 of the trauma site 200 is suboptimal.

A second solution to the issue of avoiding collisions between the OTS system 100 and items disposed within trauma site rooms 202 is omitting the OTS system 100 entirely and employing one or more mobile X-ray devices within the trauma site 200. However, this solution is also suboptimal as it adds another item that is required to be moved within a room 202 around the table 130, the other medical equipment 208 and the medical personnel already present. In this situation, during the clinical imaging process, radiologists are required to adjust the position of the X-ray devices and wallstands for each patient to achieve the desired orientation for obtaining the necessary images of the patient. However, manual positioning of the devices in the room 202 requires a significant amount of time and energy, which reduces imaging efficiency and prolongs waiting time of each user for the X-ray device. In addition, the complex arrangement of the X-ray device and other necessary medical equipment to obtain the images often causes undesired imaging mistakes as a result of mispositioning of the imaging devices and/or through distraction of the user as a result of the extensive device positioning process.

Therefore, it is desirable to develop a system and method for positioning an OTS system including an X-ray source and optionally an X-ray detector relative to a patient that overcomes these limitations of the current prior art.

### SUMMARY OF THE DISCLOSURE

According to one aspect of an exemplary embodiment of the disclosure, an imaging system includes a multiple degree of freedom overhead support system adapted to be mounted to a surface within an environment for the imaging system, an imaging device mounted to the overhead support system, a visual sensor disposed on the imaging device, a non-visual sensor disposed on the imaging device, a motion controller operably connected to the overhead support system, a processor operably connected to the motion controller, and the visual sensor and the non-visual sensor to send control signals to and to receive data signals from the overhead support system the visual sensor and the non-visual sensor, and a memory operably connected to the processor, the memory storing processor-executable instructions therein for operation of a self-navigating and positioning system configured to generate a three-dimensional (3D) map of the environment of the imaging system with visual data from the visual sensor, and non-visual data from the non-visual sensor, wherein the processor-executable instructions when executed by the processor to operate the self-navigation and positioning system cause generation of the 3D map of the environment, and navigation of the overhead support system within the environment from a start position to a finish position to avoid collisions with one or more objects identified on the 3D map within the environment.

According to still another aspect of an exemplary embodiment of the disclosure, a method for navigating an overhead support system of an imaging system through an environment includes the steps of providing an imaging system having a multiple degree of freedom overhead support system adapted to be mounted to a surface within an environment for the imaging system, an imaging device mounted to the overhead support system, a visual sensor disposed on the imaging device, a non-visual sensor disposed on the imaging device, a motion controller operably connected to the overhead support system, a processor operably connected to the motion controller, and the visual sensor and the non-visual sensor to send control signals to and to receive data signals from the overhead support system the visual sensor and the non-visual sensor, and a memory operably connected to the processor, the memory storing processor-executable instructions therein for operation of a self-navigating and positioning system configured to generate a three-dimensional (3D) map of the environment of the imaging system with visual data from the visual sensor, non-visual data from the non-visual sensor and position data from the motion controller, generating the 3D map of the environment, and navigating the overhead support system within the environment from a start position to a finish position to avoid collisions with one or more objects identified on the 3D map within the environment.

These and other exemplary aspects, features and advantages of the invention will be made apparent from the following detailed description taken together with the drawing figures.

### BRIEF DESCRIPTION OF THE DRAWINGS

The drawings illustrate the best mode currently contemplated of practicing the present invention.

In the drawings:

FIG. 1 shows a diagram of a prior art imaging system utilizing an overhead support system in a trauma site including a number of treatment rooms.

FIG. 2 is an isometric view of a trauma site having a number of treatment rooms and an imaging system including an overhead support system capable of moving an X-ray source between the treatment rooms according to an exemplary embodiment of the disclosure.

FIG. 3 is an isometric view of a room including an overhead support system with the self-navigating positioning system according to an exemplary embodiment of the disclosure.

FIG. 4 is an isometric view of an X-ray source utilized with the overhead support system and universal positioning system of FIG. 3.

FIG. 5 is schematic view of the operation of the self-navigating positioning system according to an exemplary embodiment of the disclosure.

FIG. 6 is a flowchart illustrating an exemplary method of operation of the self-navigating positioning system according to an exemplary embodiment of the disclosure.

FIG. 7 is a schematic view of a visual sensor image employed in a 2D semantic segmentation performed by the self-navigating positioning system according to an exemplary embodiment of the disclosure.

FIG. 8 is schematic view of a non-visual sensor image employed in a homogeneous coordinate transformation performed by the self-navigating positioning system according to an exemplary embodiment of the disclosure.

FIG. 9 is a schematic view of an aligned image output from a coordinate alignment step performed by the self-navigating positioning system according to an exemplary embodiment of the disclosure.

FIG. 10 is a schematic view of a 3D map output by the self-navigating positioning system according to an exemplary embodiment of the disclosure.

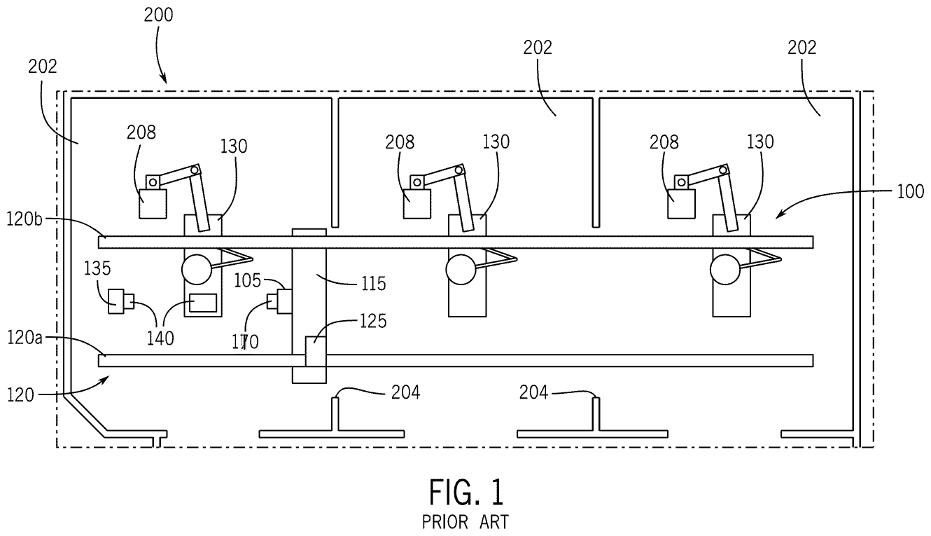

### DETAILED DESCRIPTION OF THE DRAWINGS

One or more specific embodiments will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present invention, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments. As used herein, the terms “substantially,” “generally,” and “about” indicate conditions within reasonably achievable manufacturing and assembly tolerances, relative to ideal desired conditions suitable for achieving the functional purpose of a component or assembly. Also, as used herein, “electrically coupled”, “electrically connected”, and “electrical communication” mean that the referenced elements are directly or indirectly connected such that an electrical current may flow from one to the other. The connection may include a direct conductive connection, i.e., without an intervening capacitive, inductive or active element, an inductive connection, a capacitive connection, and/or any other suitable electrical connection. Intervening components may be present. The term “real-time,” as used herein, means a level of processing responsiveness that a user senses as sufficiently immediate or that enables the processor to keep up with an external process.

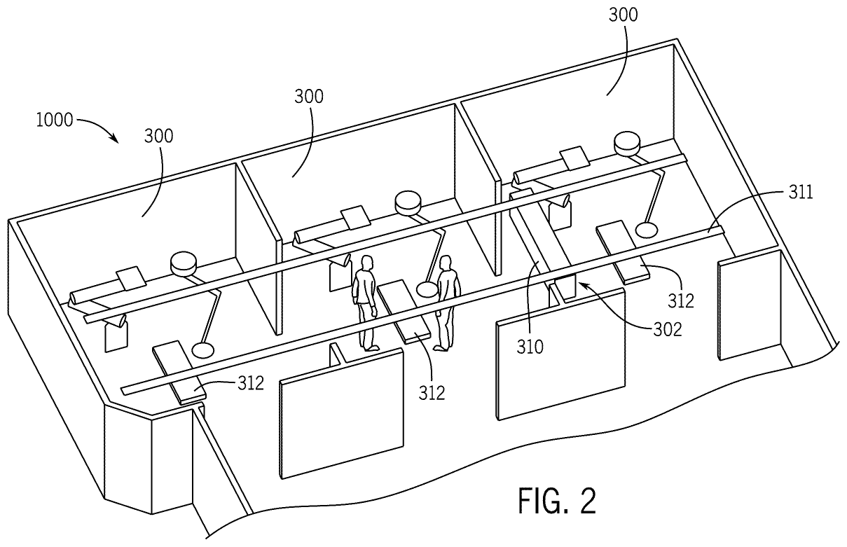

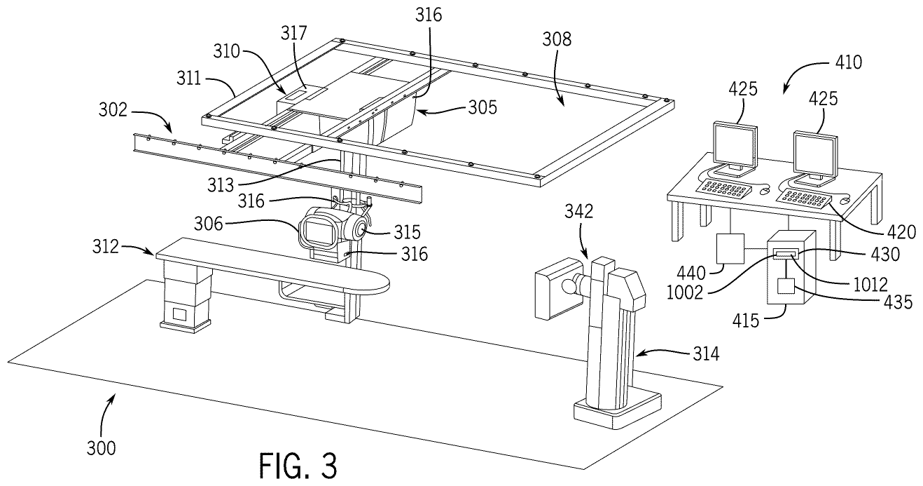

FIGS. 2 and 3 show an exemplary embodiment of a trauma site 1000 including a number of adjoining treatment rooms 300 and including an imaging system 302 that is moveable between the rooms 300 to obtain desired images of patients located in the rooms 300. The imaging system 302 includes a self-navigating and positioning system 1002 disposed on a workstation 410 for controlling the movement and operation of the various components of the imaging system 302.

The imaging system 302 is formed with a first imaging device 306, which can be an X-ray tube or a detector, that is secured by a moveable mount 305 to a portion of the site 1000 or other location in which the imaging system 302 is disposed, such as a wall or ceiling 308 of the site 300, where the moveable mount 305 can be an overhead support system 310 or a robotic arm. The individual rooms 300 each include one or more of a table 312, a wall stand 314, and a second imaging device 342, which can be the other of the X-ray tube or the detector forming the imaging system 302, and which can be incorporated within one or both of the table 312 and the wall stand 314.

The overhead support system 310, which in the illustrated exemplary embodiment supports the X-ray source as the first imaging device 306, provides five (5) separate degrees of freedom/axes of automated or manually-directed movement for the first imaging device 306, and in particular allows for lateral and longitudinal movement of the mount 305 along a suspended track 311 for the overhead support system 310, vertical movement via a telescopic column 313 attached to the mount 305 and moveable along the suspended track 311 with the mount 305, rotational movement relative to the mount 305 provided by the rotation of the column 313, and angular movement provided by a pivot mechanism 315 disposed between the column 313 and the first imaging device 306. The overhead support system 310 also includes one or more suitable position monitor(s) 316 in order to provide accurate and precise location information regarding the position of the first imaging device 306 disposed on the overhead support system 310 and the independently moveable component parts 305,313,315 of the overhead support system 310. The movement of each of the component parts 305,313,315 of the overhead support system 310 are controlled by a motion controller 317 interconnected with the overhead support system 310 and including one or more motors (not shown) or other motive devices operable to independently and selectively move the mount 305, the column 313 and the pivot mechanism 315, and an inertial measurement unit (IMU) (not shown) integrated or forming a component part therewith, which can enable the motion controller 317 to provide position data/information regarding the acceleration and angular velocity of motion of the overhead support system 310, optionally in real-time, along each axis of motion for the overhead support system 310 and/or first imaging device 306, e.g., horizontal axis-longitudinal (x) axis and lateral (y) axis, and/or vertical (z) axis, etc., as well as regarding any rotational motion.

It should also be understood that the imaging system 302 may also include other components suitable for implementing the disclosed embodiments. Exemplary imaging procedures that can be performed by the imaging system 302 includes radiography procedures such as, but not limited to, Computed Tomography (CT) procedures, computerized axial tomography (CAT) scanning procedures, and fluoroscopy procedures.

The overhead support system 310, and by extension the first imaging device 306, are operably connected to a workstation 410 which constitutes at least part of the self-navigating and positioning system 1002, which in an exemplary embodiment is disposed remotely from the imaging system 302, such as at a location outside of the rooms 300 of the trauma site 1000. The workstation 410 may include a computer 415, one or more input devices 420, for example, a keyboard, mouse, or other suitable input apparatus, and one or more output devices 425, for example, display screens or other devices providing data from the workstation 410. The workstation 410 may receive commands, scanning parameters, and other data from an operator or from a memory 430 and processor 435 of the computer 415. The commands, scanning parameters, and other data may be used by the computer 415/processor 435 to exchange control signals, commands, and data with one or more of the overhead support system 310, the first imaging device 306, the table 312, the wall stand 314, and the second imaging device 342 through a suitable wired or wireless control interface 440 connected to each of these components of the imaging system 302. For example, the control interface 440 may provide control signals to and receive image, position or other data signals from one or more of the overhead support system 310, the first imaging device 306, the table 312, the wall stand 314, and the second imaging device 342. In addition, the motion controller 317 is operably connected to the workstation 410 in order to receive information from the workstation 410 regarding the current and desired location of the overhead support system 310, and of each of the independently moveable components of the overhead support system 310, e.g., the mount 305, the column 313 and the pivot mechanism 315. In a particular exemplary embodiment, the motion controller 317 communicates with the workstation 410 through cable or wireless techniques regarding the current position, angle and desired location of the overhead support system 310.

The workstation 410 may control the frequency and amount of radiation produced by the X-ray source 306 or 342, the sensitivity of the detector 306 or 342, and the positions of the table 312 and wall stand 314 in order to facilitate scanning operations. Signals from the detector 306 or 342 may be sent to the workstation 410 for processing. The workstation 410 may include an image processing capability for processing the signals from the detector 306 or 342 to produce an output of real time 2D or 3D images for display on the one or more output devices 425. Further, with the 5 axes of motion provided by each of the overhead support system 310 and the wall stand 314, the self-navigating positioning system 1002 enables the imaging system 302 to perform classical table imaging and wall stand procedures with only a single detector 306,342. In addition, the ability of the overhead support system 310, the table 312 and the wall stand 314 to be operated automatically provides the imaging system 302 utilizing the self-navigating positioning system 1002 with the ability for the overhead support system 310 to be autonomously and/or manually controlled, such as via the workstation 410.

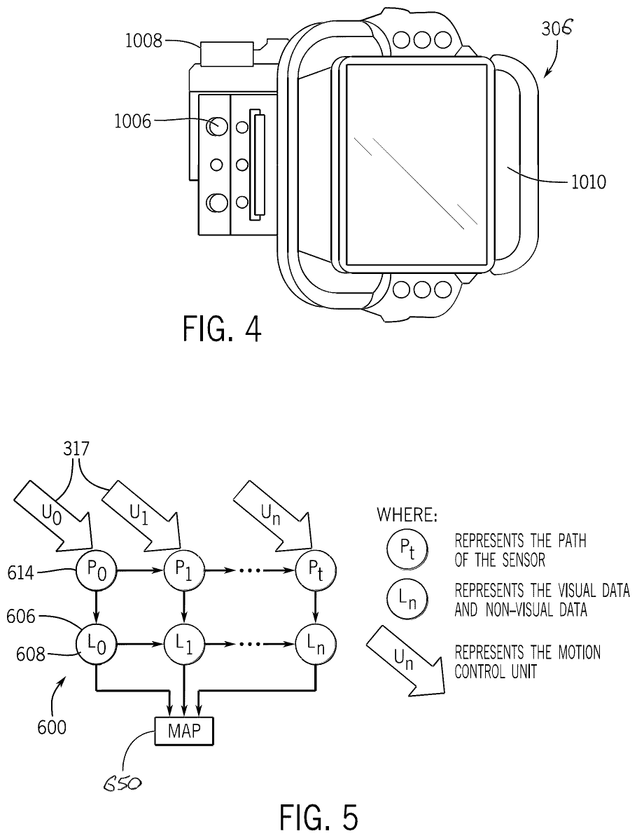

Referring now to FIG. 4, the X-ray source 306 disposed on the overhead support system 310 includes a visual sensor 1006 and a non-visual sensor 1008, each disposed on the housing 1010 of the X-ray source 306 and each capable of obtaining three-dimensional (3D) information on the environment surrounding the overhead support system 310 and the X-ray source 306. The visual sensor 1006 can take the form of a camera, such as a depth camera, while the non-visual sensor 1008 can take the form of an infrared sensor, a lidar or radar sensor, or an ultrasound sensor, among others. The visual sensor 1006 and the non-visual sensor 1008 are located adjacent one another and oriented in the same direction on the housing 1010 in order to provide the same or similar field(s) of view of the surrounding environment to both the visual sensor 1006 and non-visual sensor 1008.

To control the movement of the overhead support system 310, in an exemplary embodiment the self-navigating and positioning system 1002 is disposed on the workstation 410 as processor-executable instructions stored in memory 430 and accessible by computer 415/processor 435 for the operation of a simultaneous location and mapping (SLAM) algorithm 1012. The SLAM algorithm 1012 enables the use of the information on the location of the overhead support system 310 within the room 300 from the position sensors 316 and/or motion controller 317 in conjunction with spatial information on the environment surrounding the overhead support system 310 provided by the visual sensor 1006 and non-visual sensor 1008 to map the environment around the overhead support system 310 and autonomously direct the movement of or navigate the overhead support system 310 through the mapped environment. More specifically, the SLAM algorithm-based, self-navigating positioning system 1012 for the overhead support system 310 generates and continuously updates an internal map (FIG. 10) of the environment surrounding the overhead support system 310, i.e., the trauma site 1000/rooms 300, as a reference when moving within the environment. This map provides positions for landmark localizations, e.g., tables 312 and wallstands 314, within the environment under dynamic conditions regarding the placement of the landmarks and other items within the environment, ad enables the motion controller 317 to be operated to move the overhead support system 310 effectively to target positions while avoiding obstacles between the overhead support system 310 and the target position.

The self-navigating positioning system 1012 overcomes the issues associated with prior art a single-sensor SLAM algorithm-based systems, where the reliability of the map generated by the prior art SLAM algorithm is compromised as, when used individually, each of the visual sensor 1006 and non-visual sensor 1008 has drawbacks under certain situations. For example, the effectiveness of the visual sensor 1006 depends highly on the environment, and in particular even slight illumination changes can produce significant negative impacts. In addition, the non-visual sensor 1008 operation is significantly degraded as a result of any off-angle reflections of the energy waves employed by the non-visual sensor 1008. Therefore, to improve the accuracy of sensing and thus enhance the robustness of the 3D map garneted by the self-navigating positioning system 1002 employing the SLAM algorithm 1012, both the visual sensor 1006 and the non-visual sensor 1008 are employed to provide information to the SLAM algorithm 1012, where a fusion of the information from each sensor 1006, 1008 is performed by the self-navigation and positioning system 1002 to calibrate the data from these sensors 1006,1008 to address the computational burden of typical camera-based mapping while simultaneously alleviating issues presented by changes in illumination within the environment to provide a more accurate and reliable map of the environment for use in navigating the overhead support system 310 within the environment. In addition, when the non-visual sensor 1008 is operated to scan over a 360° range in 2D, then the mapping process performed by the SLAM algorithm 1012 will not be impacted when the overhead support system 310 is moving either backwards or forwards.

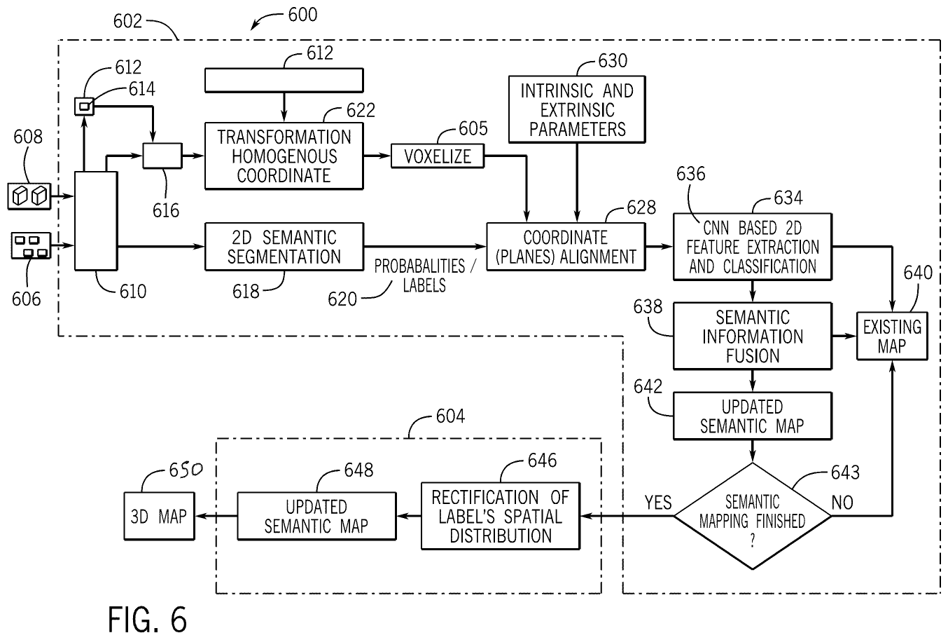

With reference now to FIG. 5, an exemplary method 600 employed by the self-navigating positioning system 1002 using the SLAM algorithm 1012 is illustrated that employs information from the motion controller 317 including visual data (i.e., 2D images)/first input 606 and the non-visual data (i.e., 3D measurement(s)/point cloud(s))/second input 608 as well as position data/motion controller data 614 on the location of the overhead support system 310 in an iterative process to generate and continuously update a map 650 of the environment to direct and control movement of the overhead support system 310 and connected imaging device 306 within the environment in real-time. In an exemplary embodiment of the method 600 shown in FIG. 6, the method 600 includes a first semantic mapping phase or operation 602 and a second map refinement phase or operation 604 to produce, e.g., in real time, a map of the environment though which the overhead support system 310 is moving, i.e., the trauma site 1000/room(s) 300, to enable the self-navigating and positioning system 1002 to move the overhead support system 310 along a path determined by the self-navigating and positioning system 1002 within the environment 1000,300 into the desired location to obtain images of a patient while avoiding collisions with other objects and/or individuals present and/or moving within the environment.

With regard to the first semantic mapping phase or operation 602, initially a first input 606 from the visual sensor 1006 and a second input 608 from the non-visual sensor 1008 are provided to the algorithm 1012 for processing into the environment map 650. In general, using the visual data/first input 606 and non-visual data/second input 608, the method 600/algorithm 1012 proceeds to:
a. synchronize the 3D measurements/point cloud(s) (e.g., second input 608) from 3D spatial/non-visual sensor 1008 to the recording time stamp recorded in association with the 2D images (e.g., first input 606) being obtained by the visual sensor 1006;
b. correct/remove motion artifacts caused by the motion of the overhead support system 310 to ensure the 3D measurements/point cloud(s) (e.g., second input 608) matches with the synchronized image plane captured by visual sensor 1006;
c. convert the 3D measurements/point cloud (second input 608) into homogenous coordinates within a homogeneous coordinate system so that the 3D measurements/point cloud(s) can align with the synchronized 2D image frames, optionally taking various intrinsic and extrinsic parameters into account:
   i. the intrinsic parameters include field of view (FOV) and focal length, among others., which are used for distortion removal to guarantee proper mapping from sensor plane to image plane.
   ii. the extrinsic parameters are positional correlations including rotation matrix and translation vector between two sensors which have different coordinate systems; with the extrinsic parameters, synchronization on sensor plane is generated according to positional relationship;
   iii. the 3D coordinates obtained from the 3D spatial/non-visual sensor 1008 will be transformed to a 2D projection based on the required parameters for alignment;
d. transform the homogenous coordinate system to Euclidean coordinate system; and
e. generate a refined 3D map 650 of using Euclidean coordinate system.

More particularly, as shown in FIGS. 6-10, in the illustrated exemplary embodiment(s) of the method 600 of operation of the SLAM algorithm 1012 of the self-navigating positioning system 1002, in the semantic mapping phase or operation of the method 600, initially in step 610 the visual data (i.e., 2D images)/first input 606 and the non-visual data (i.e., 3D measurement(s)/point cloud(s))/second input 608 are synchronized with one another, such as to match the non-visual/3D position data from the second input 608 in time with the image/visual data provided by the first input 606. In one exemplary embodiment, the synchronization can be performed by associating a time stamp recorded with the first input 606 to determine the 2D image(s) in the first input 606 obtained during the time for obtaining the 3D measurement(s)/point cloud(s) forming the second input 608. As the sampling density of the 3D sensor 1008, e.g., Lidar, is far less than that of the 2D sensor 1006, e.g., the camera, an approach to associating or registering the Lidar point cloud to the camera coordinate system according to timestamp made during frame sampling is used. Afterwards, the synchronization in step 610 results in a sparse information mapping outcome. Additionally, the synchronization in step 610 can include a third input 612 in the form of position data/motion controller data 614 regarding the position and/or direction and speed of movement of the overhead support system 310 under the direction of the motion controller 317 at the time associated with the time stamps for the image data in the first input 606.

In step 616, subsequent to the synchronization in step 610, the first input 606 data is corrected with regard to any motion artefacts in the 3D measurements/point cloud(s) forming the first input 606 resulting from the movement of the overhead support system 310 during the time the 2D image(s) were obtained. Motion artefacts, referring generally to the failure in obtaining sensor odometry information through visual approach under rapid movement of the sensor, e.g., camera, are mainly concerned in 2D image data processing. In this circumstance, the pose of the visual sensor 1006, e.g., position and orientation, is mainly calculated from continuous 2D image frames, which is a rough estimation due to the fact that consecutive frames accumulate error all the time. Particularly, when the overhead support system 310 is moving or rotating excessively fast, limited frame rate (i.e., the number of images obtained by the visual sensor 1006 in a specified period of time) results in motion blur in the 2D images/first input 606. This artefact correction can be accomplished by the algorithm 1012 in a known manner, e.g., where ineffective odometry information caused by motion artefacts is replaced by the IMU to ensure proper pose estimation for the sensor 1006, also with the information in the third input 612 from the motion controller 317 regarding the position and movement (i.e., speed and direction) of the overhead support system 310 during the time of synchronization.

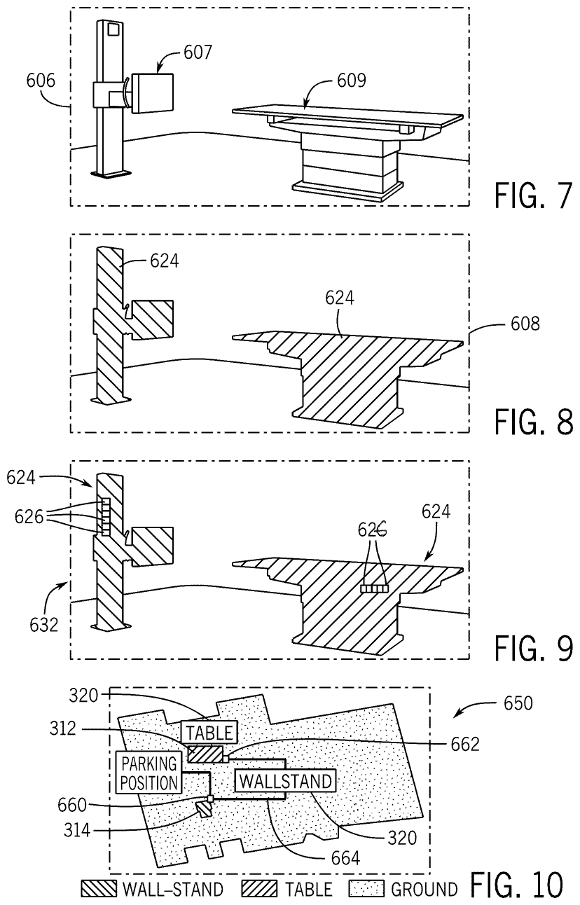

After synchronization and correction, the visual data/first input 606 from the visual sensor 1006 is processed in a 2D semantic segmentation step 618. In this step 618, as best shown in FIGS. 6 and 7, the 2D camera images/first input 606 is analyzed by the SLAM algorithm 1012, such as by an artificial intelligence (AI) 1014, such as a deep learning module (e.g., U-Net) or convolutional neural network (CNN), forming part of the SLAM algorithm 1012 that is trained to identify shapes 607, 609 of various structures within the 2D images of the environment, such as the room 300 of the trauma site 1000. In performing the semantic segmentation, the AI 1014 identifies shapes 607, 609 in the 2D images/first input 608 and classifies the shapes 607, 609 as various object and surface types present within the 2D images/first input 606 and applies labels 620, e.g., “table”, “wallstand”, “floor”, etc., to the various objects and/or surfaces identified within the 2D images/first input 608 along with a probability or confidence for each of the selected label(s) 620, as illustrated in FIG. 7.

Concurrently to the 2D semantic segmentation step, as shown in FIGS. 6 and 8, the synchronized and corrected 3D measurements/point cloud(s) forming the second input 608 are processed or transformed by the SLAM algorithm 1012 in step 622 from a homogeneous coordinate system for the 3D measurements/point cloud(s) of the second input 608, such as based on the known location of the 3D sensor 1008 via data/third input 614 from the motion controller 317 and the positions of the 3D measurements/point cloud(s) of the second input 608 relative to the non-visual sensor 1008. More specifically, in step 622, the homogenous coordinate system of the projected depth information forming the 3D measurements/point cloud(s) of the second input 608 is transformed into a globally consistent coordinate system. Each 3D measurement/point cloud is sensed by the 3D sensor 1008 and stored in a homogenous form, which can be one of a number of selectable coordinate forms for the 3D measurement/point cloud that can be stored in memory 430 to be accessed by the SLAM algorithm 1012 for post-processing purposes as defined by the sensor 1008, and then transformed in step 622 by the algorithm 1012 to a consistent (public/standard) coordinate system that can be readily combined or fused with the odometry information provided by either the motion controller 317 and/or 2D camera images/first input 606 for 3D map generation. Once the 3D measurements/point cloud(s) forming the second input 608 are defined with respect to the homogeneous coordinates in step 622, the SLAM algorithm 1012 can define/identify one or more volumes 624 using the homogeneous coordinates for the 3D measurements/point cloud(s) for each of the various component parts of the 3D measurements, i.e., the point cloud(s), of the second input 608, with each defined volume 624 being transformed in step 625 into constituent voxels 626 representing the 3D measurements/point cloud(s) of the volume(s) 624, as shown in FIG. 8. As the second input 608 can and often will contain multiple separate and/or overlapping components within the 3D measurements/point cloud(s), the volume 624 and constituent voxels 626 will represent the various objects and/or surfaces detected by the non-visual sensor 1008 and constituting the 3D measurements/point cloud(s).

After the semantic segmentation of the 2D image in step 618 and the formation of the volume(s) 624 within the homogeneous coordinate system in step 622, as shown in the exemplary embodiment of FIGS. 6 and 9, the voxelized 3D measurements/point cloud(s) 624 of the second input 608 are aligned with the labeled 2D image(s)/first input 606 in step 628 to form one or more aligned planes or images 632. In this alignment, in addition to the introduction of various intrinsic and/or extrinsic parameters 630 described previously, the voxels 626 forming the volume(s) 624 are projected/overlapped onto and aligned with the synchronized and labeled 2D image 606 corresponding to the 3D measurement(s)/point cloud(s) in the second input 608, as shown in FIG. 9. In one exemplary embodiment of the alignment process of step 628, for each frame, i.e., each view captured by the 2D visual sensor 1006 that can be matched with 3D sensor data/point cloud from the non-visual sensor 1008 where the amount of frames obtained within unit of time, i.e., frame rate described previously, the 3D measurements/point clouds of the second input 608 are projected to 2D visual plane/images of the first input 606. This projection is based on the determined association, i.e., positional relationship and timestamps, between each 2D visual plane/images of the first input 606 with the associated 3D measurement(s)/point cloud(s) of the second input 608 in order to obtain sparse alignment points with depth values. After this, an upsampling technique is applied to the sparse points to form a dense map which corresponds with the 2D image. As the semantic segmentation was made previously in step 618 on the 2D images with labels as output, voxels 626 in the upsampled map are associated with the semantic results of step 618 to assign labels on each voxel 626 containing depth information. This alignment is performed for one or more associated pairs of the synchronized 2D images 606 and 3D measurement(s)/point cloud(s) 608 to enable each volume 624 in the second input 608 to align with the labeled objects present in the synchronized 2D image 606 containing a planar view of the volume(s) 624. Further, for each 2D image/visual data obtained by the visual sensor 1006 at a particular position and/or orientation of the visual sensor 1006 within the first input 606, an alignment of the synchronized 3D spatial measurement(s)/point cloud(s) within the second input 608 can be performed to produce additional aligned images 632 of the same environment 1000,300 and the object(s) therein to form a set of aligned images 632. This set of aligned images 632 can be utilized to define a semantic 3D map 633 of the environment 1000, 300, optionally in a real-time manner as the visual sensor 1006 and non-visual sensor 1008 are moved about the environment 1000,300 by the overhead support system 310, as schematically illustrated in FIG. 5.

In conjunction with the projection and alignment of the volume(s) 624/voxels 626 onto the synchronized 2D image(s) 606, in the aligning process performed in step 628, the coordinates of the voxels 626 within the homogeneous coordinate system are also converted or transformed into the Euclidean coordinate system represented within the 2D image 606. This process is performed using the correspondence of the position of the voxels 626 in the synchronized 2D image 606 and the Euclidean coordinate system defined within the 2D image 606 of the environment 1000,300 in order to enable the semantic 3D map 640 to conform to the Euclidean coordinate system defined in the environmemtn 1000, 300.

Once the alignment in step 628 is completed, due to discrepancies in the positioning or aligning of the voxels 626 forming the volume 624 onto the representations of the objects and/or surfaces in the 2D image 606, projection errors are also introduced into the aligned images 632. To address these errors, in step 634 a separate CNN 636, such as a CNN trained to perform spatial reasoning, e.g., voxel distance clustering, is employed by the algorithm 1012 to decide which voxels 626 correspond to the actual location(s) of the object(s) in the 2D image 606, and which voxels 626 represent projection errors that are to be removed from the aligned image 632. To assist in the classification of the voxels 626 to be retained and removed, the CNN 636 analyzes the point cloud(s)/volume(s) 624 formed of the voxels 626 in order to identify and provide a label for the object(s) represented in each aligned image 632 by the point cloud(s)/volume(s) 624. Using the identification/label, the CNN 636 can remove voxels 626 located outside of expected volumes for the identified object(s), i.e., voxels 626 identified as being associated with projection errors and not with an object(s).

In step 638, the SLAM algorithm 1012 can fuse the results of the 2D image sematic segmentation in step 618 as a verification of the identification provided as the output of the CNN 636, and enables the CNN 636 to define the types and shapes of the objects represented by the volumes 624 such that only the voxels 626 aligned with the proper positions and/or shapes for the identified object(s) can be retained by the CNN 636 within the aligned image 632. This combination of the sematic segmentation and identification using each of the 2D image 606 and the aligned image 632 effectively minimizes the error in precisely locating the object(s) within the environment of the overhead support system 310, and enables the SLAM algorithm 1012 to produce a highly accurate semantic 3D map 640 of the environment 1000,300.

Further, in step 642, the SLAM algorithm 1012 can update the sematic 3D map 640, such as by providing analysis results, e.g., additional aligned images 632 of additional first inputs 606 and second inputs 608, such as first inputs 606 and second inputs 608 taken or obtained subsequent to the formation of the semantic 3D map 640, The semantic fusion analysis results from the additional first inputs 606 and second inputs 608 can be employed by the SLAM algorithm 1012 to update the semantic 3D map 640 with enhanced probabilities of the labels for different voxels 626 to more precisely define the shapes and/or locations of the objects in the environment 1000,300, optionally to provide a semantic 3D map 640 of the environment 1000,300 in real-time.

To determine the semantic 3D map 640 is complete in step 643, in one exemplary embodiment the mapping process continues through each frame obtained by the visual sensor 1006 to produce the aligned images 632 including the associated 2D semantic information from step 618 with 3D projection points until all voxels 626 in the 3D map 640 are labeled, though the labels are not necessarily completely accurate. To rectify the labels for the voxels 626, the sematic mapping phase or operation 602 is terminated and the sematic 3D map 640 is output to the map refinement phase or operation 604 of the SLAM algorithm 1012. In the refinement phase 604, initially in step 646 the spatial distribution of the labels/associated objects or volumes 624 in the semantic 3D map 640 are rectified to more clearly identify and segment the space(s) occupied by and disposed between the volumes 624 in the semantic 3D map 640, thereby providing the areas through which the overhead support system 310 can be moved from the current position to the desired imaging position. As stated previously, some semantic information is incorrect in terms of the labels for the voxels 626, which are in a relatively low density and few might stay far away from the ground truth regarding voxel clustering. By applying the spatial distribution rectification in step 646, the final semantic map 650 is generated with fewer error or mislabeled voxels 626. Therefore, a better segmentation performance is achieved in each frame for 3D mapping.

Finally, in step 648 the voxels 626 identified previously as being errors with regard to the location and shape of the labeled volumes 624 are removed from the semantic 3D map 640 in order to provide a 3D map 650, as shown in the exemplary embodiment of FIG. 10, for use by the self-navigating and positioning system 1002 to move the overhead support system 310 from the current and/or parked position 1016 to the desired imaging position 1018, e.g., adjacent the table 312, between the locations of the identified and labeled volumes 624 on the map 650. The 3D map 650 can be presented in any desired orientation, such that even in a top view of the 3D 650, a general representation is provided including the location and labels for different objects present in the environment 1000,300. The 3D map 650 is therefore more reliable for use in by the self-navigating and positioning system 1002 routing due to the ability to not only locate but identify the object(s) within the environment, thus enabling the system 1002 to distinguish between which object(s) to avoid and which are adjacent the desired position for the overhead support system 310. Further, with dynamic nature of the environment 1000,300 able to be sensed by the different sensors 1006,1008 of the system 1002 in real-time, potential collisions between he overhead support system 310 and the object(s) in the environment 1000,300 are more detectable and avoidable than in prior art collision-mitigation systems. Further, while in certain embodiments the 3D map 650 is not presented on a display 425 to the user of the imaging system 302 and utilized only internally by the self-navigating and positioning system 1002, on other embodiments the 3D map 650 can be provided on the display 425, optionally for further verification by the user of the labels for the object(s)/volume(s) 624 represented in the 3D map 650, as well as the start and/or parking position 660, finish position 662 and selected route 664 for the overhead support system 310 which can be also be selectively identified within the 3D map 650.

In addition to the ability of the self-navigating and positioning system 1002 to employ both a visual sensor 1006 and a non-visual sensor 1008 in a fusion 3D mapping process performed by a SLAM algorithm 1012 to provide highly accurate 3D maps for identification of and navigation around objects located in a surrounding environment, there are additional benefits to the self-navigating and positioning system 1002 of the present disclosure. More specifically, the use of input from two different types of sensors, e.g., visual and non-visual, allows the self-navigating and positioning system 1002 to generate a more accurate map independent of each of the illumination and scale of the environment in which the system 1002 is operated. Further, the semantic segmentation information in the 3D map 650 includes the system-defined labels 620 for each of the object(s) or component(s) present in the 3D map 650, allowing users to instantly identify the object(s) on within the 3D map 650.

In addition to the benefits provided by the self-navigating and positioning system 1002 regarding the information provided to the users and for the operation of the motion controller 317 via the 3D map 650, the real-time information obtained by the self-navigating and positioning system 1002 regarding the movement of the object(s) within the environment over time offers new potential for the reconstruction, visualization, and identification of patterns of movement within the environment. For example, with the recorded camera images and 3D spatial information, the 3D maps generated by the self-navigating and positioning system 1002 over specific time frames can illustrate the paths taken by objects and personnel within the environment covered by the 3D map 650, which can be used as clinical evidence to improve in-room workflow. In a similar manner, the 3D maps generated by the self-navigating and positioning system 1002 over specific time frames can be employed to calculate averages for time spent for each patient from the 4D data consisting of the dynamic 3D map 650 or cine or video of the 3D map 650, in conjunction with the associated timestamp.

Finally, it is also to be understood that the self-navigating and positioning system 1002 and/or the SLAM algorithm 1012 may include the necessary computer, electronics, software, memory, storage, databases, firmware, logic/state machines, microprocessors, communication links, displays or other visual or audio user interfaces, printing devices, and any other input/output interfaces to perform the functions described herein and/or to achieve the results described herein. For example, as previously mentioned, the system may include at least one processor/processing unit/computer and system memory/data storage structures, which may include random access memory (RAM) and read-only memory (ROM). The at least one processor of the system may include one or more conventional microprocessors and one or more supplementary co-processors such as math co-processors or the like. The data storage structures discussed herein may include an appropriate combination of magnetic, optical and/or semiconductor memory, and may include, for example, RAM, ROM, flash drive, an optical disc such as a compact disc and/or a hard disk or drive.

Additionally, a software application(s)/algorithm(s) that adapts the computer/controller to perform the methods disclosed herein may be read into a main memory of the at least one processor from a computer-readable medium. The term “computer-readable medium”, as used herein, refers to any medium that provides or participates in providing instructions to the at least one processor of the systems 302,304 (or any other processor of a device described herein) for execution. Such a medium may take many forms, including but not limited to, non-volatile media and volatile media. Non-volatile media include, for example, optical, magnetic, or opto-magnetic disks, such as memory. Volatile media include dynamic random access memory (DRAM), which typically constitutes the main memory. Common forms of computer-readable media include, for example, a floppy disk, a flexible disk, hard disk, magnetic tape, any other magnetic medium, a CD-ROM, DVD, any other optical medium, a RAM, a PROM, an EPROM or EEPROM (electronically erasable programmable read-only memory), a FLASH-EEPROM, any other memory chip or cartridge, or any other medium from which a computer can read.

While in embodiments, the execution of sequences of instructions in the software application causes at least one processor to perform the methods/processes described herein, hard-wired circuitry may be used in place of, or in combination with, software instructions for implementation of the methods/processes of the present invention. Therefore, embodiments of the present invention are not limited to any specific combination of hardware and/or software.

It is understood that the aforementioned compositions, apparatuses and methods of this disclosure are not limited to the particular embodiments and methodology, as these may vary. It is also understood that the terminology used herein is for the purpose of describing particular exemplary embodiments only, and is not intended to limit the scope of the present disclosure which will be limited only by the appended claims.

We claim:

1. An imaging system comprising:
a. a multiple degree of freedom overhead support system adapted to be mounted to a surface within an environment for the imaging system;
b. an imaging device mounted to the overhead support system;
c. a visual sensor disposed on the imaging device;
d. a non-visual sensor disposed on the imaging device;
e. a motion controller operably connected to the overhead support system;
f. a processor operably connected to the motion controller, and the visual sensor and the non-visual sensor to send control signals to and to receive data signals from the overhead support system the visual sensor and the non-visual sensor; and
g. a memory operably connected to the processor, the memory storing processor-executable instructions therein for operation of a self-navigating and positioning system configured to generate a three-dimensional (3D) map of the environment of the imaging system with visual data from the visual sensor, and non-visual data from the non-visual sensor,
wherein the processor-executable instructions when executed by the processor to operate the self-navigating and positioning system cause:
i. generation of the 3D map of the environment; and
ii. navigation of the overhead support system within the environment from a start position to a finish position to avoid collisions with one or more objects identified on the 3D map within the environment.

2. The imaging system of claim 1, wherein the visual sensor is a camera.

3. The imaging system of claim 1, wherein the non-visual sensor is 3D spatial sensor.

4. The imaging system of claim 3, wherein the self-navigating and positioning system is configured to generate a three-dimensional (3D) map of the environment of the imaging system with visual data from the visual sensor, non-visual data from the non-visual sensor and position data from the motion controller.

5. The imaging system of claim 1, wherein the self-navigating and positioning system includes a simultaneous localization and mapping algorithm, and wherein the processor-executable instructions when executed by the processor to operate the simultaneous localization and mapping algorithm causes:
a. performing a semantic mapping operation to generate a semantic 3D map; and
b. performing a map refinement operation to generate the 3D map from the semantic 3D map.

6. The imaging system of claim 5, wherein the processor-executable instructions when executed by the processor to perform the semantic mapping operation causes:
a. synchronizing the visual data with the non-visual data;
b. converting the non-visual data into homogenous coordinates;
c. aligning the non-visual data with the visual data;
d. converting the homogeneous coordinates of the non-visual data to Euclidean coordinates; and
e. generating the semantic 3D map from the non-visual data.

7. The imaging system of claim 6, wherein the processor-executable instructions when executed by the processor to perform the semantic mapping operation causes correction of motion artefacts in the visual data prior to aligning the non-visual data in homogenous coordinates with the visual data.

8. The imaging system of claim 6, wherein the processor-executable instructions when executed by the processor to perform the semantic mapping operation causes:
iii. semantic segmentation of the visual data to identify objects represented in the visual data and form semantic visual data prior to aligning the non-visual data with the visual data;
iv. feature extraction and classification of the non-visual data to identify objects represented in the non-visual data after aligning the non-visual data with the semantic visual data; and
v. fusion of the semantic visual data and the feature extraction and classification of the non-visual data to form a semantic 3D map used to create the 3D map.

9. The imaging system of claim 6, wherein the processor-executable instructions when executed by the processor to align the non-visual data with the visual data causes:
a. defining the non-visual data into voxels forming one or more volume(s) in the non-visual data using the homogeneous coordinates; and
b. overlaying the voxels of the non-visual data onto the semantic visual data.

10. The imaging system of claim 9, wherein the processor-executable instructions when executed by the processor to convert the homogeneous coordinates of the non-visual data to Euclidean coordinates causes converting the homogeneous coordinates of the voxels to Euclidean coordinates.

11. The imaging system of claim 9, wherein the processor-executable instructions when executed by the processor to extract and classify features of the non-visual data to identify objects represented in the non-visual data causes:
a. labeling the one or more volumes within the non-visual data formed by the voxels; and
b. removing voxels located outside of the volumes.

12. The imaging system of claim 1, wherein the imaging device is an X-ray tube.

13. A method for navigating an overhead support system of an imaging system through an environment, the method comprising the steps of:
a. providing an imaging system comprising:
i. a multiple degree of freedom overhead support system adapted to be mounted to a surface within an environment for the imaging system;
ii. an imaging device mounted to the overhead support system;
iii. a visual sensor disposed on the imaging device;
iv. a non-visual sensor disposed on the imaging device;
V. a motion controller operably connected to the overhead support system;
vi. a processor operably connected to the motion controller, and the visual sensor and the non-visual sensor to send control signals to and to receive data signals from the overhead support system the visual sensor and the non-visual sensor; and
vii. a memory operably connected to the processor, the memory storing processor-executable instructions therein for operation of a self-navigating and positioning system configured to generate a three-dimensional (3D) map of the environment of the imaging system with visual data from the visual sensor, non-visual data from the non-visual sensor and position data from the motion controller,
b. generating the 3D map of the environment; and
c. navigating the overhead support system within the environment from a start position to a finish position to avoid collisions with one or more objects identified on the 3D map within the environment.

14. The method of claim 13, wherein the self-navigating and positioning system includes a simultaneous localization and mapping algorithm, and wherein the method includes the steps of:
a. performing a semantic mapping operation using the simultaneous localization and mapping algorithm to generate a semantic 3D map; and
b. performing a map refinement operation to generate the 3D map from the semantic 3D map.

15. The method of claim 14, wherein the step of performing the semantic mapping operation causes:
a. synchronizing the visual data with the non-visual data;
b. converting the non-visual data into homogenous coordinates;
c. aligning the non-visual data with the visual data;
d. converting the homogeneous coordinates of the non-visual data to Euclidean coordinates; and
e. generating the semantic 3D map from the non-visual data.

16. The method of claim 15, wherein the step of performing the semantic mapping operation comprises correcting motion artefacts in the visual data prior to aligning the non-visual data in homogenous coordinates with the visual data.

17. The method of claim 15, wherein the step of performing the semantic mapping operation comprises:
a. semantic segmentation of the visual data to identify objects represented in the visual data prior to aligning the non-visual data with the visual data;
b. extracting and classifying features of the non-visual data to identify objects represented in the non-visual data after aligning the non-visual data with the visual data; and
c. fusing the semantic segmentation of the visual data and the feature extraction and classification of the non-visual data to form a semantic 3D map used to create the 3D map.

18. The method of claim 15, wherein the step of aligning the non-visual data with the visual data causes:
a. defining the non-visual data into voxels forming one or more volume(s) in the non-visual data using the homogeneous coordinates; and
b. overlaying the voxels of the non-visual data onto synchronized visual data.

19. The method of claim 18, wherein the step of converting the homogeneous coordinates of the non-visual data to Euclidean coordinates comprises converting the homogeneous coordinates of the voxels to Euclidean coordinates.

20. The method of claim 19, wherein the step of extracting and classifying features of the non-visual data to identify objects represented in the non-visual data comprises:
d. labeling the one or more volumes within the non-visual data formed by the voxels; and
e. removing voxels located outside of the volumes.
